(19) United States
(12) Patent Application Publication
(10) Pub. No.: US 2024/0325736 A1
(43) Pub. Date: Oct. 3, 2024

(54) IMPLANTABLE MEDICAL LEAD WITH DISTAL BIAS

(71) Applicant: ZOLL MEDICAL CORPORATION, Chelmsford, MA (US)

(72) Inventors: Randy W. Westlund, St. Paul, MN (US); Mark C. Lynn, St. Anthony, MN (US)

(21) Appl. No.: 18/620,264

(22) Filed: Mar. 28, 2024

Related U.S. Application Data
(60) Provisional application No. 63/455,592, filed on Mar. 30, 2023.

(51) Int. Cl. A61N 1/05; A61N 1/375

(52) U.S. Cl. CPC A61N 1/057; A61N 1/37516

(57) ABSTRACT

An implantable medical electrical lead includes a lead body having a longitudinal axis, the lead body including proximal and distal portions, the proximal portion being generally straight, and the distal portion being formed with a helix having a central axis; and a plurality of electrodes arranged along the helix, each of the electrodes having a length parallel to a lead body portion on which each of the electrodes is disposed, where the longitudinal axis and the central axis are approximately coaxial. The helix is formed with a predetermined bias, and may include at least two coils with electrodes, and optionally a third coil to provide a stabilization function. A method for implanting the lead includes inserting the lead in a lumen, and positioning the lead to dispose the plurality of electrodes against an internal wall of the lumen, in order to stimulate an adjacent structure, such as the phrenic nerve.

### CROSS-REFERENCE TO RELATED APPLICATIONS

This application claims priority under 35 U.S.C. § 119 (e) to U.S. Provisional Patent Application Ser. No. 63/455,592 titled “IMPLANTABLE MEDICAL LEAD WITH DISTAL BIAS,” filed Mar. 30, 2023, the entire contents of which is incorporated herein by reference for all purposes.

### TECHNICAL FIELD

Embodiments of the present disclosure are directed to an implantable medical lead with a distal bias.

### BACKGROUND

Electrical stimulation and/or sensing leads have been implanted in veins, arteries, and other lumens of the human body to provide electrical stimulation and/or sensing functions. In addition to providing stimulation directly to the heart or spinal column, leads have provided indirect cardiac stimulation via a transvenous approach using veins in anatomically adjacent target locations. This includes applications such as cardiac resynchronization therapy (CRT), CRT with defibrillation (CRT-D), pacing, as well as other applications.

Transvenous leads present certain advantages, such as the ability to install therapy leads using a minimally invasive procedure and thus reduction of post-surgical trauma or complications. Transvenous leads have been successfully implanted in patients to provide phrenic nerve stimulation for treating central sleep apnea (CSA). Typically, the therapy stimulates either the left or right phrenic nerve, and stimulation of either phrenic nerve is configured to contract the diaphragm via pathways in the phrenic nerve leading to a full breath. Through this mechanism, ventilation is stabilized, and patients may transition into normal breathing. While the implantation of transvenous leads allows benefits over surgically implanted electrodes, implantation may present certain challenges. For example, difficulties with lead implantation may present issues with lead migration and orientation of a lead and electrodes.

### SUMMARY

According to some embodiments of the present disclosure, an implantable medical electrical lead includes: a lead body defining a longitudinal axis, the lead body having a proximal portion and a distal portion, the proximal portion being generally straight, and the distal portion being formed with a helix, the helix having a central axis being defined through the helix; and a plurality of electrodes arranged along the helix, each of the electrodes having a length parallel to a lead body portion on which each of the electrodes is disposed, where the longitudinal axis and the central axis may be approximately coaxial. The distal portion of the implantable medical electrical lead may include the helix formed with a predetermined bias. The helix of the implantable medical electrical lead may include a plurality of coils.

The lead may be selected in advance from a plurality of leads based on a diameter of a lumen in which the lead is configured to be implanted. The plurality of leads may include a small lead having a helical diameter of about 14 to 18 mm, a medium lead having a helical diameter of about 19 to 23 mm, and a large lead having a helical diameter of about 24 to 28 mm. The plurality of leads may include a lead in which coils of the helix are preformed to have a substantially constant diameter, a lead in which the coils of the helix are preformed to have an increasing diameter in a direction of a distal end of the implantable lead body, and a lead in which the coils of the helix are preformed to have a decreasing diameter in the direction of the distal end of the implantable lead body. The lead body may have a diameter of about 1.6 mm to 2.15 mm.

The lead body of the implantable medical electrical lead may include a transition portion arranged between the proximal portion and the distal portion of the lead body. The transition portion may be connected to a first coil of a plurality of coils of the helix of the distal portion. The transition portion of the implantable medical electrical lead may have a radius of curvature of about 9 mm to 11 mm. At least one of the electrodes of the plurality of electrodes may be arranged in the transition portion.

The plurality of electrodes of the implantable medical electrical lead may be arranged only in the distal portion of the lead body. For example, a first electrode of the plurality of electrodes may be fixed to a first coil of a plurality of coils of the helix. The first electrode may be arranged at about 135 to 180 degrees of the first coil, or the first electrode may be arranged at about 270 to 315 degrees of the first coil. Further, at least the first electrode and a second electrode of the plurality of electrodes may be arranged on the first coil. The first and second electrodes may be spaced apart by about 2 mm to 10 mm. In addition, each of the first and second electrodes may have a surface area of about 9 to 40 mm², preferably about 20 to 30 mm².

In the implantable medical electrical lead, the helix may be preformed with at least a first coil and a second coil. The first coil may include at least two electrodes of the plurality of electrodes. Also, the second coil may include at least two additional electrodes of the plurality of electrodes. Further, the first coil and the second coil may have a substantially constant diameter that is set to correspond to a diameter of a lumen in which the implantable medical electrical lead is configured to be implanted. Preferably the first coil includes a first electrode, a second electrode, and a third electrode, and the second coil includes a fourth electrode, a fifth electrode, and a sixth electrode.

In the implantable medical electrical lead, the helix may be preformed with at least a first coil, a second coil, and a third coil. The first coil may include at least two electrodes of the plurality of electrodes, where the second coil includes at least two additional electrodes of the plurality of electrodes, and the third coil is configured as a stabilization coil without including any electrodes. Alternatively, the first coil may be configured as a stabilization coil without including any electrodes, the second coil may include at least two electrodes of the plurality of electrodes, and the third coil may include at least two additional electrodes of the plurality of electrodes.

In the implantable medical electrical lead, the helix may be formed in a generally circular shape. Alternatively, the helix may be formed in a generally oval shape.

In the implantable medical electrical lead, the distal portion may define a bias region in which coils of the helix are preformed to have a substantially constant diameter. Alternatively, the distal portion may define a bias region in which coils of the helix are preformed to have an increasing diameter in a direction of a distal end of the implantable lead body, where the helix is preformed with at least a first coil, a second coil, and a third coil. In some embodiments, the first coil may be configured as a stabilization coil without including any electrodes, the second coil may include at least two electrodes of the plurality of electrodes, and the third coil may include at least two additional electrodes of the plurality of electrodes. Alternatively, the first coil may include at least two electrodes of the plurality of electrodes, the second coil may include at least two additional electrodes of the plurality of electrodes, and the third coil may be configured as a stabilization coil without including any electrodes.

In the implantable medical electrical lead, the distal portion may define a bias region in which coils of the helix are preformed to have a decreasing diameter in a direction of a distal end of the implantable lead body, where the helix is preformed with at least a first coil, a second coil, and a third coil, the first coil includes at least two electrodes of the plurality of electrodes, the second coil is configured as a stabilization coil without including any electrodes, and the third coil includes at least two additional electrodes of the plurality of electrodes.

In the implantable medical electrical lead, the longitudinal axis of the lead body may diverge from the central axis of the helix by an angle α such that the longitudinal axis and the central axis are approximately coaxial, where the angle α is less than 25 degrees. Alternatively, the longitudinal axis of the lead body and the central axis helix may be substantially parallel to one another and displaced from one another by less than a radius of a coil of the helix, such that the longitudinal axis and the central axis are approximately coaxial.

According to some embodiments of the present disclosure, a method for implanting a medical electrical lead includes steps of: inserting the lead in a lumen, the lead including: a lead body defining a longitudinal axis, the lead body having a proximal portion and a distal portion, the proximal portion being generally straight, and the distal portion being formed with a helix, the helix having a central axis being defined through the helix; a plurality of electrodes arranged along the helix, each of the electrodes having a length parallel to a lead body portion on which each of the electrodes is disposed, where the longitudinal axis and the central axis may be approximately coaxial; and positioning the lead within the lumen such that the plurality of electrodes are disposed against an internal wall of the lumen. The distal portion of the implantable medical electrical lead may include the helix formed with a predetermined bias. The helix of the implantable medical electrical lead may include a plurality of coils.

The method may further include selecting the lead in advance based on a diameter of the lumen in which the lead is to be implanted. The selecting step may include selecting from a plurality of leads of different helical diameters. The plurality of leads may include a small lead having a helical diameter of about 14 to 18 mm, a medium lead having a helical diameter of about 19 to 23 mm, and a large lead having a helical diameter of about 24 to 28 mm. The small lead, the medium lead, or the large lead may be selected based on a diameter of the lumen in which the lead is implanted. The plurality of leads may include a lead in which coils of the helix are preformed to have a substantially constant diameter, a lead in which the coils of the helix are preformed to have an increasing diameter in a direction of a distal end of the implantable lead body, and a lead in which the coils of the helix are preformed to have a decreasing diameter in the direction of the distal end of the implantable lead body. The method may further include conducting venous imaging to assess a diameter of the lumen in which the lead is to be implanted. The step of conducting venous imaging may include using a venogram or ultrasound to image the lumen.

In conjunction with the method, the lead body of the implantable medical electrical lead may include a transition portion arranged between the proximal portion and the distal portion of the lead body. The transition portion is connected to a first coil of a plurality of coils of the helix of the distal portion. The transition portion of the implantable medical electrical lead may have a radius of curvature of about 9 mm to 11 mm. At least one of the electrodes of the plurality of electrodes may be arranged in the transition portion.

In conjunction with the method, the plurality of electrodes of the implantable medical electrical lead may be arranged only in the distal portion of the lead body. For example, a first electrode of the plurality of electrodes may be fixed to a first coil of a plurality of coils of the helix. The first electrode may be arranged at about 135 to 180 degrees of the first coil, or the first electrode may be arranged at about 270 to 315 degrees of the first coil. Further, at least the first electrode and a second electrode of the plurality of electrodes may be arranged on the first coil. The first and second electrodes may be spaced apart by about 2 mm to 10 mm. In addition, each of the first and second electrodes may have a surface area of about 9 to 40 mm², preferably about 20 to 30 mm².

In conjunction with the method, the helix of the lead may be preformed with at least a first coil and a second coil. The first coil may include at least two electrodes of the plurality of electrodes. Also, the second coil may include at least two additional electrodes of the plurality of electrodes. Further, the first coil and the second coil may have a substantially constant diameter that is set to correspond to a diameter of a lumen in which the implantable medical electrical lead is configured to be implanted. Preferably the first coil includes a first electrode, a second electrode, and a third electrode, and the second coil includes a fourth electrode, a fifth electrode, and a sixth electrode.

In conjunction with the method, the helix of the lead may be preformed with at least a first coil, a second coil, and a third coil. The first coil may include at least two electrodes of the plurality of electrodes, where the second coil includes at least two additional electrodes of the plurality of electrodes, and the third coil is configured as a stabilization coil without including any electrodes. Alternatively, the first coil may be configured as a stabilization coil without including any electrodes, the second coil may include at least two electrodes of the plurality of electrodes, and the third coil may include at least two additional electrodes of the plurality of electrodes.

In conjunction with the method, the helix of the lead may be formed in a generally circular shape. Alternatively, the helix may be formed in a generally oval shape.

In conjunction with the method, the distal portion of the lead may define a bias region in which coils of the helix are preformed to have a substantially constant diameter. Alternatively, the distal portion may define a bias region in which coils of the helix are preformed to have an increasing diameter in a direction of a distal end of the implantable lead body, where the helix is preformed with at least a first coil, a second coil, and a third coil. In some embodiments, the first coil may be configured as a stabilization coil without including any electrodes, the second coil may include at least two electrodes of the plurality of electrodes, and the third coil may include at least two additional electrodes of the plurality of electrodes. Alternatively, the first coil may include at least two electrodes of the plurality of electrodes, the second coil may include at least two additional electrodes of the plurality of electrodes, and the third coil may be configured as a stabilization coil without including any electrodes.

In conjunction with the method, the distal portion of the lead may define a bias region in which coils of the helix are preformed to have a decreasing diameter in a direction of a distal end of the implantable lead body, where the helix is preformed with at least a first coil, a second coil, and a third coil, the first coil includes at least two electrodes of the plurality of electrodes, the second coil is configured as a stabilization coil without including any electrodes, and the third coil includes at least two additional electrodes of the plurality of electrodes.

According to some embodiments of the present disclosure, an implantable medical electrical lead includes: a lead body defining a longitudinal axis, the lead body having a proximal portion and a distal portion, the proximal portion being generally straight, and the distal portion being formed with a helix, the helix being preformed with a plurality of coils and having a central axis being defined through the helix; and a plurality of electrodes arranged along the helix, each of the electrodes having a length parallel to a lead body portion on which each of the electrodes is disposed, where a first coil of the plurality of coils includes at least two electrodes of the plurality of electrodes, and a second coil of the plurality of coils includes at least two additional electrodes of the plurality of electrodes, where the longitudinal axis and the central axis may be approximately coaxial.

The first coil and the second coil of the implantable medical electrical lead may have a substantially constant diameter that corresponds to a diameter of a lumen in which the implantable medical electrical lead is to be implanted.

In the implantable medical electrical lead, the first coil may include a first electrode, a second electrode, and a third electrode, and the second coil may include a fourth electrode, a fifth electrode, and a sixth electrode.

According to another aspect of the present disclosure, an implantable medical electrical lead kit is provided. The implantable medical electrical lead kit comprises a plurality of implantable medical electrical leads, each respective implantable medical electrical lead of the plurality of implantable medical electrical leads having a lead body that includes a proximal portion and a distal portion, the proximal portion being generally straight and the distal portion being formed with a helix, the helix including a plurality of electrodes arranged along the helix, each respective electrode having a length parallel to a lead body portion on which the respective electrode is disposed. The distal portion of each respective implantable medical electrical lead of the plurality of implantable medical electrical leads has at least one of a different shape of the helix, a different diameter of the helix, a different number of coils of the helix, or a different placement of the plurality of electrodes on the coils of the helix than any other implantable medical electrical lead of the plurality of implantable medical electrical leads comprising the implantable medical electrical lead kit.

In accordance with one embodiment, each respective implantable medical electrical lead of the plurality of implantable medical electrical leads has a different shape of the helix than any other implantable medical electrical lead of the plurality of implantable medical electrical leads comprising the implantable medical electrical lead kit. In accordance with this embodiment, the plurality of implantable medical electrical leads may include a first implantable medical electrical lead having a substantially constant diameter helix, a second implantable medical electrical lead having an increasing diameter helix in a direction of a distal end of the lead body, and a third implantable medical electrical lead having a decreasing diameter helix in the direction of the distal end of the lead body.

In accordance with another embodiment, each respective implantable medical electrical lead of the plurality of implantable medical electrical leads has a different diameter of the helix than any other implantable medical electrical lead of the plurality of implantable medical electrical leads comprising the implantable medical electrical lead kit. In accordance with this embodiment, the plurality of implantable medical electrical leads may include a first small diameter implantable medical electrical lead having a helical diameter of about 14 to 18 mm, a second medium diameter implantable medical electrical lead having a helical diameter of about 19 to 23 mm, and a third large diameter implantable medical electrical lead having a helical diameter of about 24 to 28 mm.

In accordance with yet another embodiment, each respective implantable medical electrical lead of the plurality of implantable medical electrical leads has a different number of coils of the helix than any other implantable medical electrical lead of the plurality of implantable medical electrical leads comprising the implantable medical electrical lead kit. In one embodiment, the plurality of implantable medical electrical leads may include a first implantable medical electrical lead that includes two coils in the helix and a second implantable medical electrical lead that includes three coils in the helix.

In accordance with a further embodiment, each respective implantable medical electrical lead of the plurality of implantable medical electrical leads includes three coils in the helix, wherein a placement of the plurality of electrodes on each respective implantable medical electrical lead is different than any other implantable medical electrical lead of the plurality of implantable medical electrical leads comprising the implantable medical electrical lead kit. In accordance with this embodiment, the plurality of implantable medical electrical leads may include a first implantable medical electrical lead in which the plurality of electrodes are disposed on only the first and second coils of the helix, a second implantable medical electrical lead in which the plurality of electrodes are disposed on only the second and third coils of the helix, and a third implantable medical electrical lead in which the plurality of electrodes are disposed on only the first and third coils of the helix.

According to another aspect of the present disclosure, an implantable medical electrical lead is provided that comprises: a lead body defining a longitudinal axis, the lead body having a proximal portion and a distal portion, the proximal portion being generally straight, and the distal portion being formed with a helix, the helix having a central axis being defined through the helix; and a plurality of electrodes arranged along the helix, each of the electrodes having a length parallel to a lead body portion on which each of the electrodes is disposed, wherein the longitudinal axis and the central axis are approximately coaxial. In some embodiments, the longitudinal axis may diverge from the central axis by an angle α, such that the longitudinal axis and the central axis are approximately coaxial, wherein a is less than 25 degrees. In other embodiments, the longitudinal axis and the central axis are substantially parallel to one another and displaced from one another by less than a radius of a coil of the helix, such that the longitudinal axis and the central axis are approximately coaxial.

In various embodiments of the implantable medical electrical lead, the lead body comprises a transition portion arranged between the proximal portion and the distal portion of the lead body, where the transition portion has a radius of curvature of about 9 mm to 11 mm. In some embodiments, the transition portion has a length along the central axis of about 20 mm. In at least one embodiment, the transition portion comprises a proximal segment comprising a first curve in a first plane, a distal segment comprising a second curve in a second plane, and an intermediate segment disposed between the proximal segment and the distal segment and comprising a complex curve in multiple planes. In some embodiments, the helix comprises a plurality of coils, and the radius of curvature of the transition portion is not less than a radius of curvature of a smallest coil of the helix.

### BRIEF DESCRIPTION OF THE DRA WINGS

Various aspects of and examples are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1 is a side perspective view of an example lead according to some embodiments of the present disclosure.

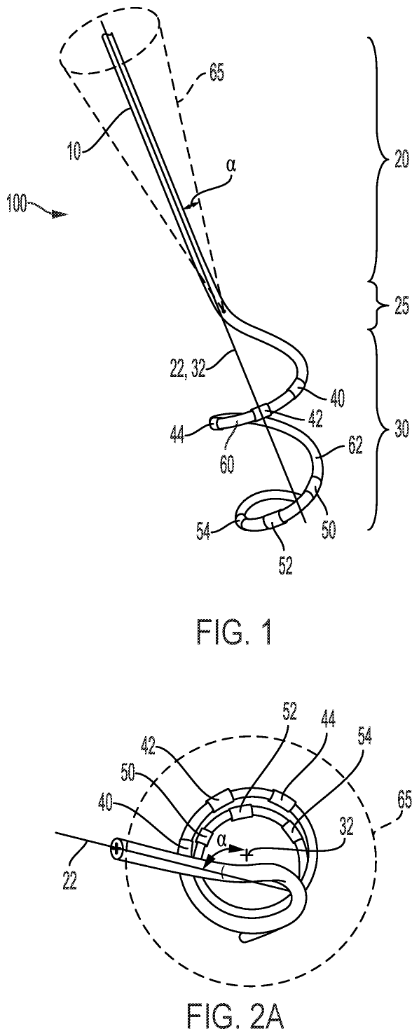

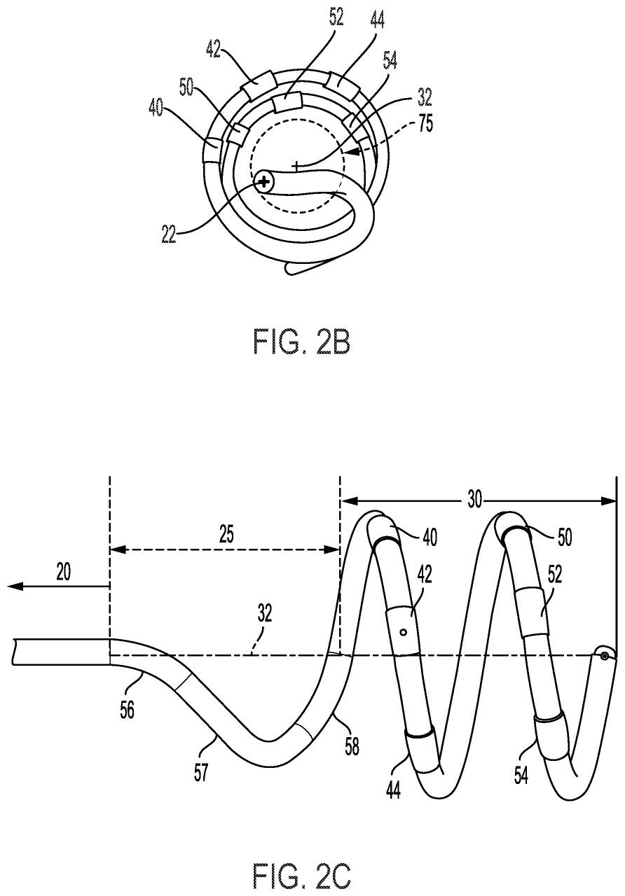

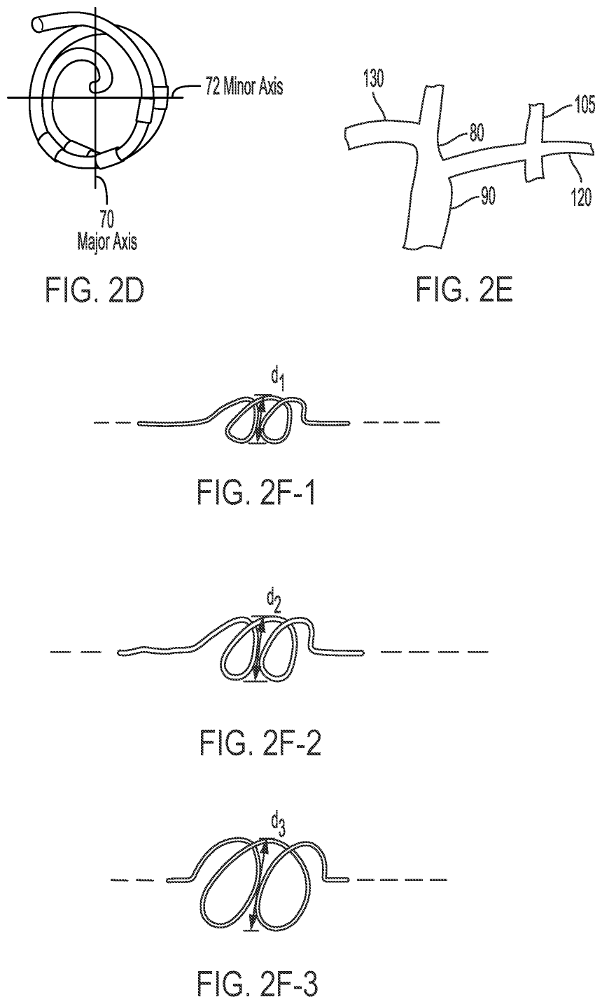

FIG. 2A is a top perspective view of the example lead of FIG. 1.

FIG. 2B is an alternative top perspective view of the example lead of FIG. 1.

FIG. 2C is an enlarged side perspective view of the distal portion of the example lead of FIG. 1.

FIG. 2D is a bottom perspective of the example lead of FIG. 1.

FIG. 2E is a perspective view of an example anatomical arrangement of selected veins of a patient.

FIGS. 2F-1, 2F-2, and 2F-3 are schematic views of example leads having different diameters.

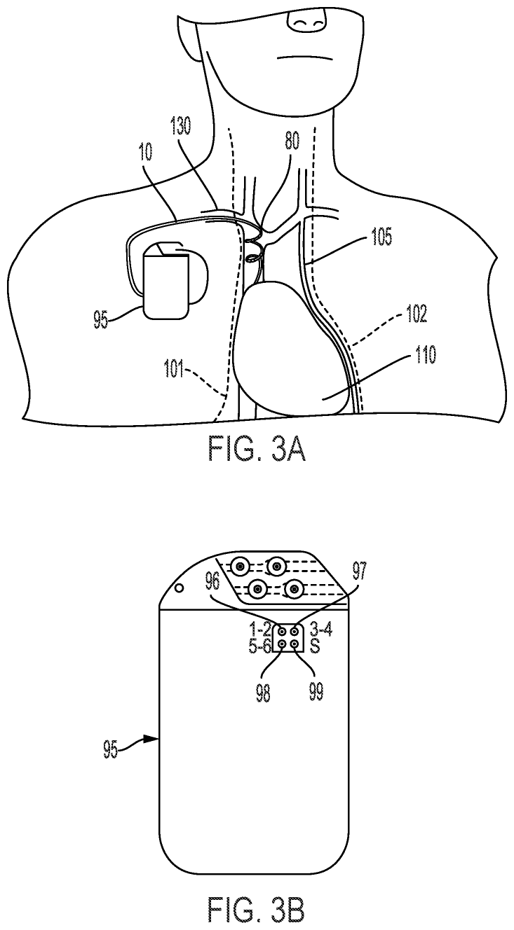

FIG. 3A is a perspective view of an example lead connected to an implantable pulse generator (IPG) in an implanted state according to some embodiments.

FIG. 3B is a schematic view of an example IPG suitable for implantation according to some embodiments.

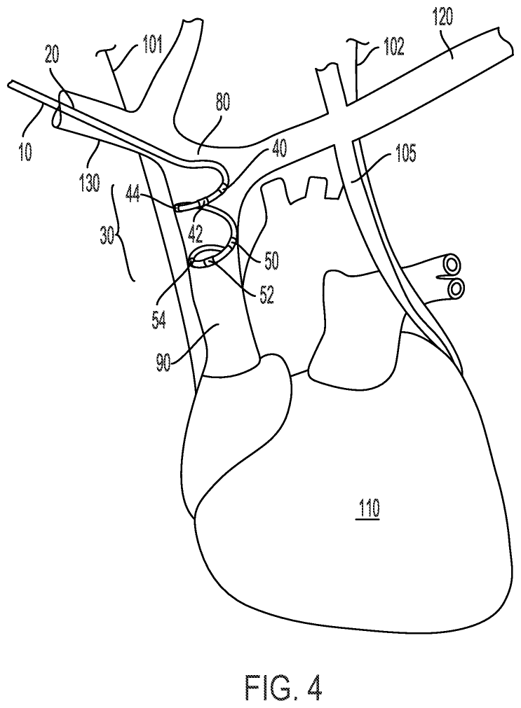

FIG. 4 is a perspective view of an example lead in an implanted state according to some embodiments.

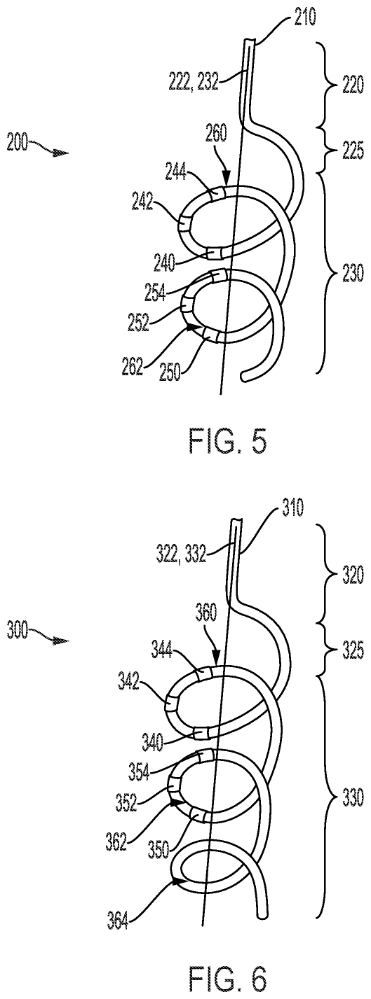

FIG. 5 is a perspective view of an example lead having two coils according to some embodiments.

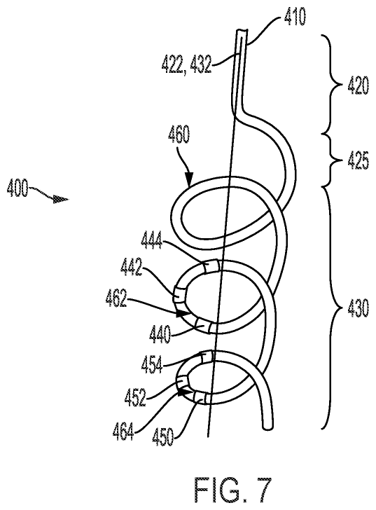

FIGS. 6 and 7 are perspective views of example leads having three coils according to some embodiments.

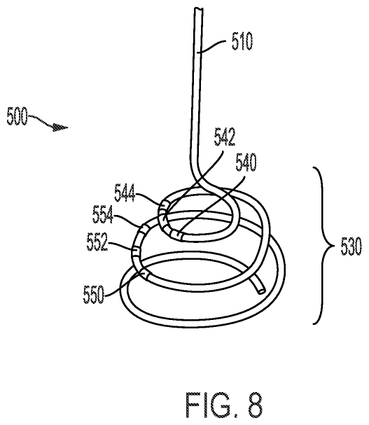

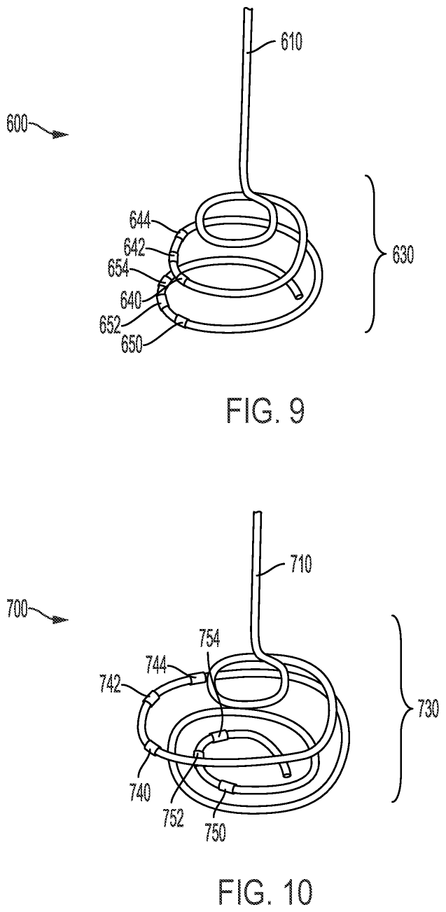

FIGS. 8 and 9 are perspective views of example leads having three coils in which the coils diverge in a distal direction of each lead.

FIG. 10 is a perspective view of an example lead having three coils in which the coils converge in a distal direction of the lead.

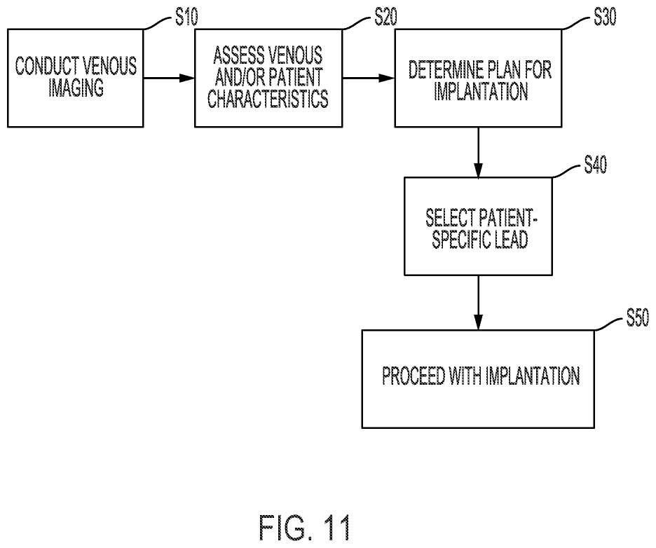

FIG. 11 is a flow diagram of a method of selecting an example patient-specific lead according to some embodiments.

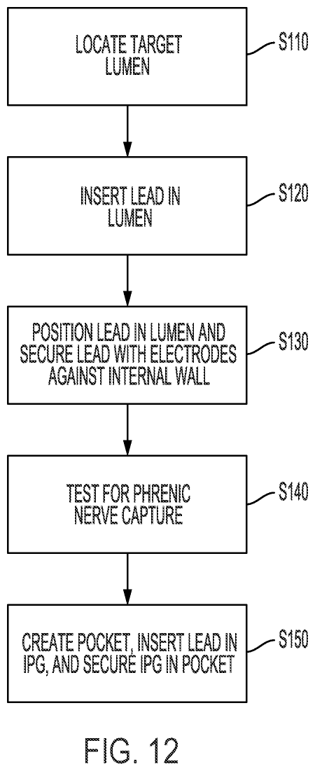

FIG. 12 is a flow diagram of a method for implanting an example lead according to some embodiments.

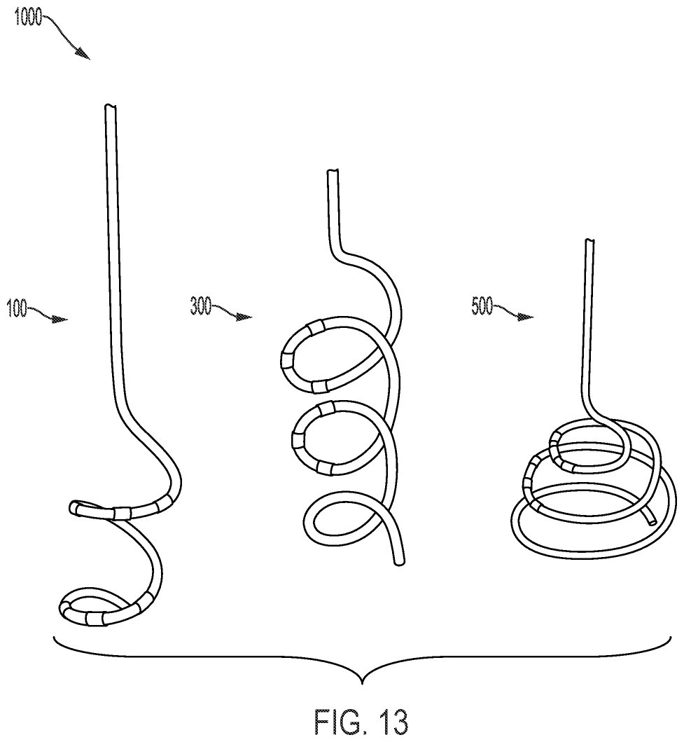

FIG. 13 illustrates a lead kit that can a plurality of differently sized and/or shaped leads from which a physician or other trained medical professional may select an appropriate patient-specific lead.

### DETAILED DESCRIPTION

Systems, methods, and devices according to the present disclosure incorporate medical devices that are configured to deliver stimulation and/or sense physiological conditions in the human body. A system may include an implantable pulse generator (IPG) and transvenous leads, for example a lead for stimulation of a patient's phrenic nerve and a lead to sense respiration via transthoracic impedance (TI). The TI respiration signal may be measured by either the sensing lead, which may be placed in the azygos vein, or the stimulation lead.

An example lead according to the present disclosure may be a stimulation lead, preferably a transvenous, stylet delivered, non-steroid eluting lead for stimulation of the phrenic nerve, and which may also be configured to sense transthoracic impedance when the lead is placed in a thoracic vein and connected with a compatible pulse generator (i.e., the IPG). The stimulation lead has an elongated lead body with a longitudinal axis, which contains a proximal portion and a distal portion. The lead further includes electrodes distributed along the distal portion of the lead body. The stimulation lead may include a plurality of electrodes, for example in certain embodiments the lead may include four electrically active ring electrodes (referred to as a “quadripolar” arrangement), and in other embodiments it may include six electrically active ring electrodes (referred to as a “hexapolar” arrangement). Further, the distal portion of the lead has a helical shape and includes a plurality of helical coils. The stimulation leads of the present disclosure may be configured to be placed in the left pericardiophrenic vein or the right brachiocephalic vein, which are anatomically adjacent to the left and right phrenic nerves, respectively. In certain cases, for example when placement in the right brachiocephalic vein is difficult or impossible, the stimulation lead may be placed in the upper portion of the superior vena cava in order to stimulate the right phrenic nerve.

In the leads of the present disclosure, the proximal portion of the lead body may be straight or substantially straight, and the distal portion may be formed into a plurality of helical coils with a predetermined bias of different diameters. The helix at the distal portion of the lead may be described as having a central axis being defined through the helix. A plurality of electrodes may be arranged along one or more of the helices, each of the electrodes having a length parallel to a lead body portion on which each of the electrodes is disposed. In certain embodiments the distal portion of the lead may comprise two coils, with at least two electrodes arranged on each of the coils. In other embodiments the helix may comprise three coils, with at least two electrodes arranged on two of the three coils and the third coil may not contain any electrodes. This additional third coil may provide stability when implanted in a patient's vasculature. For example, by providing a third coil in the helix of the lead body, which may be desired in certain patients (depending upon the patient's vascular anatomy) the lead can more easily be retained at a desired location in the vein, and the lead is less likely to become dislodged during normal movements of the patient. In certain patients, the third coil may be necessary to provide additional lead surface area (or lead body) that is in contact with the vessel walls and/or additional radial force. The additional lead surface area and/or additional radial force may provide improved stability after implantation. In addition, in embodiments where the additional third coil is positioned between the proximal portion of the lead and distal coils (which contain the electrodes), the additional third coil may help isolate the distal stimulation coils from movement of the proximal lead body. In additional embodiments, the distal portion of the lead may have more than three coils, if additional coils are required to provide enhanced stabilization in particular patients. The coils may have substantially constant diameters ranging from about 15 mm to 28 mm, and the helix may be formed in a generally circular or oval shape.

In addition to the helical coils, the lead body may further include a transition portion arranged between the proximal and distal portions, adjacent to a first coil (of the plurality of coils). At least one of the electrodes may be arranged in the transition portion. By providing electrode(s) in the transition portion, it is possible to enhance electrode coverage, for example, by providing one or more electrodes located distally from the proximal portion but before the distal, helical portion of the lead body. Such electrodes provided in the transition portion may be used to sense respiration, and provide a sensing vector having less artifacts from the cardiac cycle as compared to electrodes provided in the helix. Further, electrodes in the transition portion could function as anode(s) or cathode(s) for stimulation of the phrenic nerve. Alternatively, the electrodes may be arranged only in the distal, helical portion of the lead body, for example in at least two of the coils.

The leads of the present disclosure are preferably configured to comprise approximately coaxial proximal and distal portions in order to prevent undesirable movement or strain, for example, whipping, during implantation. In particular, the proximal portion of the disclosed leads forms a relatively straight line along the longitudinal axis, which approximately coincides with the central axis of the helix, thus providing an approximately coaxial relationship between the proximal and distal portions of the lead. As a result, it is possible to prevent whipping during deployment of the lead. Whipping may occur when rotation is applied to a lead body, but a distal portion (e.g., a helix) does not rotate at the same speed as a proximal portion of the lead body. For example, during whipping, multiple rotations (e.g., three rotations) may be applied to a proximal portion of the lead body, but the rotation may not translate to the distal portion of the lead body, such that the distal helix or helices may not rotate by the same number of rotations (e.g., they may undergo less than the three rotations, possibly even one rotation or less than one rotation). In such a condition, torque may build up in the lead body, such that when the torque is sufficient to overcome the resistance of the distal portion, the helix in the distal portion may move suddenly and spin in an uncontrolled manner. When whipping occurs in this manner, the implantation becomes challenging, as the excess movement of the lead coils makes it difficult to control and orient electrodes on the lead body within a lumen, i.e., a vein such as the right brachiocephalic vein. According to the present disclosure, the central axis of the helix is approximately coaxial with a remainder of the lead body (i.e., the generally straight proximal portion), which allows the distal portion to rotate in a one-for-one manner with the proximal portion, thus minimizing whipping during deployment.

A stimulation lead according to the present disclosure incorporates lead materials and components that have a high tensile strength and resistance to mechanical abrasion, for example, polyurethane (a synthetic, segmented polymer with high tensile strength and resistance to mechanical abrasion) or other memory-based materials such as shape-memory polymers (SMPs), shape memory alloys (SMAs), etc. Such materials present certain advantages in lead construction. For example, when the lead is constructed from such materials, it allows for a thinner layer of insulation to be used to cover lead conductors, thereby reducing overall lead diameter. Also, polyurethane has excellent lubricity and handling characteristics, with a low frictional coefficient, which can facilitate implantation of lead systems by decreasing the physical interactions between the leads. According to the present disclosure, the outer insulation layer of the leads may comprise materials that are inert and/or biostable over extended periods of time, for example, polyurethane, silicone, silicone rubber (polysiloxane). Additionally, the outer insulation layer may comprise a flexible material, such as silicone, which may decrease the risk of internal damage, including lead perforation. Silicone in particular is also more thermally resistant to the effects of electrocautery, which can be a significant advantage during pulse generator replacement or system revision. In particular embodiments, the lead body may incorporate polyurethane tubing, and portions of the lead may incorporate silicone.

Also, as provided herein, the leads each include an IS-1 compatible proximal end terminal pin or connector. In particular, IS-1 compatibility falls under an international standard (i.e., ISO 5841) regarding pacing leads of the low-profile in-line type. Electrodes according to the present disclosure may be made of a platinum-iridium (Pt/Ir) alloy. Although the leads/components are described as incorporating the above materials, other types of materials are considered to fall within the scope of the present disclosure.

A method of forming the lead in a desired shape may include use of an oven and a forming mandrel. The lead body may be formed into a desired shape by wrapping the lead body around a mandrel and subsequently exposing the assembly to a heat forming process in which the proximal and distal portions are exposed to a desired temperature. For example, in one such process, a mandrel is placed inside the lead body and/or the lead body is restrained around tooling to plastically deform the metallic portions of the lead body (typically a conductor coil) in a desired shape. This assembly of the lead body with mandrel received therein and/or tooling may next be placed in the oven for a predetermined amount of time at a predetermined temperature so that the polymer portion of the lead body retains the form of the desired shape. Once the assembly is removed from the oven and allowed to cool, and the mandrel and/or the tooling are removed from the lead body, the lead body retains the desired shape.

The above-described method of forming the lead in the desired shape may be incorporated into a production process for leads according to the present disclosure. For example, the production process may include assembling individual electrodes on the lead body, wherein the individual electrodes may be formed of multiple filars.

FIG. 1 illustrates an example of an implantable medical electrical lead 100 (hereinafter referred to as a “lead”) according to some embodiments of the present disclosure. The lead 100 has a lead body 10 including a proximal portion 20 that is generally straight, and a distal portion 30 that is formed into a helix. The distal portion 30 (i.e., the helix) preferably is formed with a predetermined bias, which results in a spiral and/or corkscrew appearance when in its relaxed state. For example, the distal portion 30 may be formed by wrapping a shape memory material, such as polyurethane, around a spindle, mandrel or other type of tooling and heat treating or otherwise shaping the lead body such that the lead body 10 is biased to return to its preformed shape (for example after implantation in a patient's vasculature). The lead body may incorporate polyurethane tubing and/or silicone, and the lead may include an IS-1 compatible proximal end terminal pin or connector. Other types of materials may be suitable for use with the lead and/or components of the lead.

As shown in FIGS. 2A-2D, the shape of the bias of the helix may be circular (e.g., FIGS. 2A-2C) or oval (e.g., FIG. 2D). For example, in some embodiments (e.g., FIG. 2D), a major axis 70 of the helix may have a diameter of about 15 to 29 mm, and a minor axis 72 of the helix may have a diameter of about 11 to 25 mm. As illustrated in FIGS. 2A through 2C, a plurality of electrodes 40, 42, 44, 50, 52, 54 may be disposed on the distal portion 30 of the lead within only a portion of the circumference of the helix. In an implementation, all of the plurality of electrodes 40, 42, 44, 50, 52, 54 may be disposed in a same portion of the circumference of the helix.

Referring to FIG. 2E, the leads of the present disclosure may be advanced through the subclavian vein 130 and implanted in the right brachiocephalic vein 80. The leads may alternatively be implanted so as to further extend into the superior vena cava (“SVC”) 90.

Patient anatomy varies from patient to patient, including the size and shape of a patient's vasculature. Variations in the diameter and shape of a patient's lumen or lumens can make lead implantation and fixation challenging. The leads of the present disclosure provide advancements in implantation and fixation by customizing lead size and shape to provide a plurality of leads of different shapes and sizes corresponding to a variety of patient anatomies.

Patient-specific leads of the present disclosure may be designed to vary the lead helix diameter, or lead shape, or both. For example, referring to FIGS. 2F-1, 2F-2, and 2F-3, a small diameter lead, a medium diameter lead, or a large diameter lead, respectively, may be designed and provided. In addition, as described further in FIGS. 5 through 10 below, the leads may have a distal portion with a substantially constant diameter helix, a diverging diameter helix or, alternatively, a converging diameter helix. Thus, advantageously, patient-specific leads may be designed to provide a versatile selection of leads, comprising: small substantially constant diameter leads, small diameter diverging leads, small diameter converging leads, medium substantially constant diameter leads, medium diameter diverging leads, medium diameter converging leads, large substantially constant diameter leads, large diameter diverging leads, and large diameter converging leads. The patient-specific lead embodiments described herein provide advantages in improving implantation and fixation across patients of different sizes, weights, age, and diversity of vasculature.

Referring again to FIGS. 2F-1, 2F-2, and 2F-3, these figures depict a small diameter helix along a lead, a medium diameter helix along a lead, and a large diameter helix along a lead, respectively. In FIG. 2F-1, the lead may have a diameter d1 of about 14 to 18 mm. In FIG. 2F-2, the lead may have a diameter d2 of about 19 to 23 mm. In FIG. 2F-3, the lead may have a diameter d3 of about 24 to 28 mm. The diameter (d1, d2, or d3) of the lead refers to a diameter of at least one coil of a substantially constant diameter helix formed at a distal end of the lead body. With respect to a converging or diverging helix, the diameter of the lead refers to a diameter of a smallest coil of the converging or diverging helix.

Referring back now to FIG. 1, the lead body 10 has a longitudinal axis 22, and includes the proximal portion 20 that is generally straight. The distal portion 30 includes the helix having a central axis 32, where the helix includes a plurality of coils, e.g., a first coil 60 and a second coil 62. The central axis 32 of the helix may be approximately coaxial with the longitudinal axis 22 of the lead body 10. As discussed in more detail further below, it is preferred that the central axis of the helix 32 be coaxial with the longitudinal axis 22 of the lead body. However, in some implementations, in order to accommodate the diameter and flexibility of the lead body, and the manner in which the shape of the lead is formed, it is sufficient that the longitudinal axis 22 of the proximal portion 20 of the lead body deviates from the central axis 32 of the helix by an angle α such that the two axes are approximately coaxial. This is depicted in FIG. 1 by the periphery of cone 65 forming an angle α with the central axis 32 of the helix. The angle α may be about 25 degrees or less. A perspective view of this aspect is depicted in FIG. 2A. Alternatively, and as shown in the top perspective view of FIG. 2B, in some implementation, the longitudinal axis 22 of the proximal portion 20 of the lead body 10 and the central axis 32 of the helix may be parallel or substantially parallel with one another, but slightly offset from one another, such that the longitudinal axis 22 of the proximal portion of the lead body 10 lies within a cylinder 75 centered about the central axis 32 of the helix, and such that the two axes are approximately coaxial. The helix of FIG. 1 is depicted with two coils, but in other embodiments, the helix may include one or more coils or two or more coils.

A transition portion 25 may be provided as an intermediate portion (i.e., a transition) between the proximal portion 20 and the distal portion 30. For example, the transition portion 25 may be in the form of a tapered section of the lead body that transitions from the proximal portion 20 to the first coil 60 of the helix that defines the distal portion 30. The transition portion 25 preferably has a radius of curvature of about 9 to 11 mm. Optionally, at least one electrode may be arranged in the transition portion 25.

FIG. 2C is an enlarged view of the distal portion 30 of the lead 10 illustrating details of the transition portion 25. In the embodiment illustrated in FIG. 2C the distal portion has a distal length (e.g., along the central axis 32 of the helix) of about 25 mm, although in other embodiments in which the coils are more loosely spaced, this length may be about 35 mm. It should be appreciated that depending on the spacing between coils and the number of coils (e.g., 2 or 3), this distance may vary. In some implementations, this distance may be between 15-45 mm.

In accordance with an aspect of the present disclosure, the transition portion 25 includes a plurality of segments including a proximal segment 56, a distal segment 58, and an intermediate segment 57 disposed between the proximal segment 56 and the distal segment 58. The proximal segment 56 is disposed adjacent to the proximal portion 20 of the lead body 10 and comprises a simple curve in a single plane. The distal segment 57 is disposed adjacent to the distal portion 30 of the lead body 10 and again comprises a simple curve in a single plane. The intermediate segment 57 comprises a complex curve in multiple planes. In various embodiments, the length of the transition portion 25 along the central axis 32 of the helix is about 20 mm+5 mm, and the transition portion 25 has a radius of curvature of about 9-11 mm. These dimensions reflect an optimization of various design criteria that are desirable in a lead. With respect to the radius of curvature, it is preferable that the radius of curvature of the transition portion 25 is no less than the radius of curvature of the smallest coil of the helix, and preferably greater than the radius of curvature of the smallest coil, as smaller radii may damage the lead 100 or lead to increased wear and fatigue. While a larger radius of curvature of the transition portion 25 increases the durability of the lead 100, it also makes placement of the distal portion 30 in a desired location within a lumen of the patient more difficult. Applicant has determined that a transition zone length of about 20 mm and a radius of curvature of about 9-11 mm provides a durable lead that can be readily positioned in a desired location within a lumen of the patient.

According to some embodiments of the present disclosure, a plurality of electrodes is arranged along the helix, where each of the electrodes preferably has a length parallel to a lead body portion on which each of the electrodes is disposed. For example, each of the electrodes preferably has a length of about 3 to 5 mm, or more preferably about 4 mm, and an outer diameter of about 2.2 to 2.4 mm, or more preferably about 2.32 mm. Therefore, in one example, the lateral surface area of each of the electrodes may be calculated as about: π(2.32 mm)×(4 mm)=29.15 mm².

As shown in FIG. 1, the distal portion 30 includes a plurality of coils (loops) that make up the helix, including the first coil 60 and the second coil 62. In certain embodiments, the helix may include two coils (see, e.g., FIGS. 1 and 2A-2D), whereas in other embodiments, the helix may include three coils (see, e.g., FIGS. 6-10). In embodiments in which the helix includes three coils, electrodes may be present on two of the coils, and a third coil preferably is formed without electrodes, thus providing a stabilizing feature. While possible to implant a lead having a helix with more than three coils, it is preferable to limit the helix to between two and three coils, in order to avoid any elevated risk to the patient that may occur when extracting a lead having a helix with an extended length of more than three coils.

Referring to FIGS. 1, 2A-2D, and 5 in each example of a lead, the helix of the distal portion of the lead body includes two coils or three coils each having a substantially constant diameter. Such leads including a helical distal portion with a substantially constant diameter may be preferred for implantation in a lumen also having a substantially constant diameter, for example, the right or left brachiocephalic vein, rather than placement at a juncture or two different diameter lumens, such as a juncture of the right brachiocephalic vein and the SVC. When the helix provided in the distal portion of the lead body includes two coils, a plurality of electrodes is arranged on each of the coils to provide stimulation of an adjacent phrenic nerve. When the distal portion of the lead body includes three coils, the plurality of electrodes are typically arranged on only two of the three coils, and a third coil is provided for stability during the implantation process, and thus preferably does not include any electrodes. It should be appreciated that in certain embodiments, when an electrode is not being used to provide stimulation, it may be used to sense physiological parameters, such as thoracic impedance.

In the above examples of leads having two or three coils in the helix, each coil of the helix may include at least two electrodes, preferably three electrodes, which may be substantially equally spaced apart from each other. By arranging the electrodes in a spaced-apart manner, when the lead is deployed the one or more electrodes can be suitably arranged along each of the coils within a lumen such as a vein, e.g., the right brachiocephalic vein, so as to be optimally positioned to stimulate a nerve (e.g., the right phrenic nerve) located adjacent to the vein. In particular, one or more electrodes may be activated so as to provide stimulation to the phrenic nerve. Preferably any two or three electrodes may be activated to provide stimulation.

According to the example shown in FIG. 1, the first coil 60 includes first, second, and third electrodes 40, 42, and 44 (of the plurality of electrodes), and the second coil 62 includes fourth, fifth, and sixth electrodes 50, 52, and 54 (of the plurality of electrodes), although each of the coils may include more or fewer electrodes. For example, each coil may include between one and six electrodes, preferably at least two electrodes, and more preferably, three electrodes. In some embodiments, the electrodes are arranged only in the distal portion 30 of the lead body 10. For example, as shown in FIG. 1, the electrodes 40, 42, 44, 50, 52, and 54 are arranged only in the distal portion 30 of the lead body 10. In the example of FIG. 1, the first electrode 40 preferably is fixed to the first coil 60 at about 135 to 180 degrees of the first coil 60. The electrodes on each coil (e.g., the electrodes 40, 42, and 44 on the first coil 60) preferably are equally spaced apart from each other. For example, the electrodes 40, 42, and 44 may be spaced apart by about 2 mm to 10 mm. As illustrated in FIG. 1, all of the plurality of electrodes in each coil may be disposed on only a portion of the coil, such as one half or one quarter of the circumference of the coil, rather than being distributed about the entire circumference of the coil. Such a placement of the electrodes may allow more of the plurality of electrodes to be positioned on the side of the lumen proximate the adjacent phrenic nerve.

As described above, the lateral surface area of a cylinder is calculated using the equation: π×diameter×length. In one example, each of the electrodes preferably has a length of about 3 to 5 mm, or more preferably about 4 mm, and an outer diameter of about 2.2 to 2.4 mm, or more preferably about 2.32 mm. Therefore, in one example, the lateral surface area of each of the electrodes may be calculated to be about: π×(2.32 mm)×(4 mm)=29.15 mm². The electrodes may have dimensions such that a suitable range of surfaces areas is about 9 to 40 mm², more preferably, about 20 to 30 mm². The surface areas of the electrodes described herein are not limiting, and are a function of the relative lengths of the electrodes. According to the present disclosure, one or more electrodes is activated to provide stimulation of the phrenic nerve. For example, a pair of electrodes including one of the electrodes 40, 42, or 44 of the first coil 60 and one of the electrodes 50, 52, or 54 of the second coil 62 may be activated to deliver stimulation current via the lead. Typically, the pair of electrodes to be activated corresponds to the electrodes situated in the vein at locations closest to the phrenic nerve. Therefore, it is likely that one of the electrodes 40, 42, or 44 of the first coil 60 and one of the electrodes 50, 52, and 54 of the second coil 62 will be selected as the pair of electrodes to provide stimulation to the phrenic nerve.

The perspective views of FIGS. 2A through 2D illustrate that the plurality of electrodes 40, 42, 44, 50, 52, 54 may be disposed on less than the entire circumference of a coil of the helix, for example less than half the circumference, and that the helix may be circular (FIGS. 2A-2C) or shaped as an oval (FIG. 2D). In FIGS. 2A-2D, the coils of the helix are substantially the same size, although as discussed below, the coils may alternatively converge or diverge along a distal direction of the lead.

FIGS. 3A and 3B are views of an implantable pulse generator (IPG) 95 according to some embodiments, where FIG. 3A depicts the IPG 95 connected to the lead body 10 in an implanted state in a patient. Referring to FIGS. 3A and 3B, a proximal end of the lead body 10 is configured to be connected to one or more ports 96, 97, 98, and/or 99 of the IPG 95 capable of delivering stimulation to the phrenic nerve. Referring to FIG. 3B, the port 96 corresponds to electrodes #1-2, the port 97 corresponds to electrodes #3-4, the port 98 corresponds to electrodes #5-6, and the port 99 corresponds to an optional sensing electrode S. The proximal end of the lead body 10 may include three IS-1 compatible lead terminal pins, or alternately a single in-line style connector with multiple electrical contacts, that correspond to pairs of electrodes at the distal end of the lead body 10. Typically, the lead body 10 includes two or three pairs of electrodes, such that the lead terminal pins at the proximal end may be connected to the ports 96, 97, and/or 98. In addition, the port 99 is configured to receive a lead terminal pin of the optional sensing electrode S. Alternatively, the optional sensing electrode S may be replaced by an additional pair of electrodes for providing stimulation. In other embodiments, the IPG 95 may include additional or fewer ports, and the ports may be compatible with IS-1 or another type of connector.

The IPG 95 is configured to monitor the patient's respiratory signals and provide electrical stimulation to the phrenic nerve according to some embodiments of the disclosure. The IPG 95 may include electrical circuitry components and a battery, which are hermetically sealed in a titanium case. Operation of the IPG 95 is supported via telemetry using a touch screen tablet computer or similar device that enables configuration of programmable parameters, initiation of system tests, and review of diagnostic data. The IPG 95 preferably is compatible with multipolar IS-1 or non-standard in-line transvenous stimulation leads, and optionally with IS-1 bipolar sensing leads. For example, as shown in FIG. 3B, the ports 96-99 preferably are 3.2 mm connector ports that are compatible with IS-1 terminal pins, where each IS-1 lead terminal pin may be secured to a respective connector port via two set screws. Alternatively, in other embodiments, the IPG 95 may be replaced by an IPG with a single in-line connector port compatible with leads having an in-line terminal pin/connector, where the IPG with the single in-line connector port is configured to be connected to a stimulation lead and may or may not use a separate sensing lead.

Referring to FIG. 3A, during implantation, the lead body 10 is advanced through the subclavian vein 130 and disposed in the right brachiocephalic vein 80 for stimulation of the right phrenic nerve 101. Alternatively, the lead body 10 may be advanced through the subclavian vein 120 for stimulation of the right phrenic nerve and disposed in the right brachiocephalic vein 80 or may be further extended to be disposed, partially or fully, in the superior vena cava 90.

Typically, a stylet is introduced into the lead as a stiffening agent and used to guide deployment of the lead body 10 through the subclavian vein 130 and into the right brachiocephalic vein 80, when the lead is provided for stimulation of the right phrenic nerve 101. After deployment of the lead, the lead may be tested for phrenic nerve capture, and thereafter, the lead is positioned with electrodes adjacent to an internal wall of the vein, and the lead is secured within the vein. In particular, a first ligature sleeve may be positioned immediately proximal to a point of venous access and secured using permanent, non-absorbable sutures, where the ligature sleeve may be anchored to the fascia or other suitable subcutaneous tissue using permanent, non-absorbable sutures. In addition, a second suture sleeve may be placed a minimum of 10 cm proximal to the first suture sleeve with a strain relief loop between the first and second sleeves for stability. Preferably the stylet is maintained within the lumen of the stimulation lead while securing the ligature sleeve to the lead body and anchoring to tissue in order to prevent damage to the stimulation lead insulation and conductor coil.

FIG. 4 of the present disclosure depicts a lead in an implanted state within one or more veins of a patient. In the implanted state of FIG. 4, the distal portion 30 of the lead body 10 has been advanced through the subclavian vein 130 such that it is disposed in the right brachiocephalic vein 80 and may extend into the superior vena cava 90. The transition from the right brachiocephalic vein 80 to the superior vena cava 90 is imprecise, and any description in this disclosure of “right brachiocephalic vein” refers to the right brachiocephalic vein and/or the superior vena cava. Any of the plurality of electrodes (e.g., the electrodes 40, 42, 44, 50, 52, or 54) is configured to stimulate the right phrenic nerve 101, which is located proximate to the right brachiocephalic vein 80. In the illustration of FIG. 4, electrodes 44 and 54 are most proximate the phrenic nerve 101 and may be preferred as stimulation electrodes. As provided herein, the helix formed in the distal portion 30 may have a substantially constant (i.e., consistent) coil diameter throughout the length of the helix, or the helix may comprise a diverging coil diameter (wherein the diameter increases from proximal to distal end), or a converging coil diameter (wherein the diameter decreases from proximal end to distal end).

During the implantation process, the distal portion 30 of the lead body 10 is introduced into the right brachiocephalic vein 80, as shown in FIG. 4. For example, in some embodiments, the lead is a hexapolar, transvenous, stylet delivered lead. The distal portion 30 of the lead body 10 has a helical shape bias and may include six ring electrodes and a non-electrically active tip having a helical shape bias. Although the helical shape bias is illustrated in the drawing figures of this disclosure as spiraling distally toward the right (i.e., being right-handed), it should be appreciated that in other embodiments, the helical shape bias may spiral distally toward the left. The lead is configured for use with a stylet to temporarily remove the bias during implantation into the desired target vein or lumen, e.g., the right brachiocephalic vein 80. In certain embodiments, there may be three IS-1 pins/connectors on a proximal end of the lead for connecting to a compatible implantable pulse generator (IPG), and this connection may be secured using a plurality of screws, e.g., six setscrews. In other embodiments, there may be four IS-1 pins/connectors on the proximal end of the lead for connecting to the IPG. In other embodiments, there may be an in-line terminal pin/connector that is secured to the compatible IPG using various contacts and a single setscrew.

Once the lead has been implanted in the target vein, e.g., the right brachiocephalic vein 80, therapy can be delivered to one or more electrodes in a programmable manner. For example, a touch screen tablet computer external to the patient may be used to communicate with the IPG via inductive telemetry to allow for configuration of programmable settings, initiation of system testing and review of diagnostic data. Other communication devices may be used instead of a tablet computer, e.g., a desktop computer, a laptop computer, a mobile device, etc., and the communication device may be preloaded with proprietary software and operated in conjunction with an external programming wand connected to the programmer via a USB cable. For example, in some embodiments, placement of the wand over the implanted device allows for telemetry communication. The above arrangement can be used to transvenously stimulate one phrenic nerve based on one or more of the following parameters: posture and activity of the patient, time of day (typical sleep time), confirmation of intact device operation via lead impedance, not to exceed a programmable daily dose of therapy. There are two main modes of stimulation therapy: asynchronous and synchronous. In asynchronous mode, the therapy provides stimulation pulses at a fixed rate through the sleep period, typically seven or more hours. When programmed in asynchronous mode, a stimulation pulse train may be delivered to the lead at a set rate, typically referred to as a “respiration rate,” which is a programmable parameter. Alternatively, programming can be carried out in synchronous mode, in which a stimulation pulse train that drives the diaphragm is triggered by a sensed respiratory cycle.

The leads of present disclosure may be configured to provide stimulation of the right phrenic nerve. The stimulation of the right phrenic nerve contracts the right hemi-diaphragm via the efferent pathways in the nerve leading to a full breath (negative inspiratory pressure in the thoracic cavity and subsequent airflow into the lung). The device is programmed to increase stimulation amplitude within programmed parameters and use the sensed respiratory signals to assess capture of the patient's breathing. By delivering therapy as described herein, the patient's ventilation can be stabilized, and the patient can be transitioned into normal breathing.

The shape of the bias of the helix of the distal portion 30 in some embodiments is circular or oval. The circular and oval helical shapes exert force against the inner luminal surface of the vein creating a radial frictional force that retains or fixes the lead in the vein. This radial frictional force is created by forming a shape or bias in the lead that has a slightly larger diameter than a vessel diameter of the vein in which it is to be disposed. This relationship can be described in some embodiments as a bias diameter to vessel diameter ratio of between about 1.1:1 and 1.3:1. The bias diameter in these embodiments is slightly larger because some of the larger veins in which these embodiments are implanted are fairly malleable and these veins do not significantly constrain the leads, resulting in bias orientations upon implantation that are substantially similar to the unconstrained position of the helix prior to implantation. Leads with such circular or oval shaped helices may be more stably implanted in certain patients, especially where the patient's vasculature is likely to enlarge upon an increase in venous volume. For example, veins within the thorax are subject to intrathoracic pressures caused by diaphragmatic contraction. When the diaphragm contracts, it creates negative intrathoracic pressures that cause veins within the thorax to dilate, increasing venous volume and enlarging the cross-sectional area of the vein. In addition, there is a downward translation of the heart 110 and lungs that can stretch and elongate the superior vena cava 90 and the right and left brachiocephalic veins 80, 120. Pulsing from the cardiac cycle also can be translated to the superior vena cava 90 and the right and left brachiocephalic veins 80, 120.

The bias of the helix of the distal portion 30 can be formed with a substantially constant diameter helix, a diverging diameter helix, or a converging diameter helix. For example, if the lead is intended for the junction of the subclavian vein 130 and the right brachiocephalic vein 80, a converging helix may provide superior fixation and electrode contact. In other examples, the targeted site involves the brachiocephalic vein junction or superior vena cava, and in such cases, the diverging helix may be preferred. In each case the varying diameters of the coils may provide more intimate contact with the vessel wall for better fixation and electrode contact. The most proximal portions of the helix (the first ½ to full revolution) may serve as a mechanism of decoupling the most proximal portions of the helix from the more distal portions. This feature may decouple external forces (e.g., arm, shoulder, etc. motion) from the electrodes. It may also permit the bias of the helix of the distal portion 30, i.e., that part that engages the vein, to move, where such motion comes from respiration, coughing, sneezing, cardiac impulse, etc., independent of the proximal portion 20.

Referring to FIG. 5, an example is shown with an alternate electrode arrangement, as compared to FIG. 1. The lead 200 illustrated in FIG. 5 has a lead body 210 including a proximal portion 220 that is generally straight, and a distal portion 230 formed with a helix, where the distal portion 230 (i.e., the helix) may be formed with a predetermined bias. Similar to the lead 100 of FIG. 1, a central axis 232 of the helix may be approximately coaxial with a longitudinal axis 222 of the lead body 210, with the helix having a substantially constant diameter. As similarly discussed above, the longitudinal axis 222 of the lead body 210 may deviate from the central axis 232 of the helix by an angle α such that the two axes are approximately coaxial. As also similarly discussed above, the longitudinal axis 222 and the central axis 232 of the helix may be parallel or approximately parallel with one another, but slightly offset from one another, such that the longitudinal axis 222 lies within a cylinder 75 centered about the central axis 232 of the helix, and such that the two axes are approximately coaxial.

As shown in FIG. 5, a first coil 260 includes electrodes 240, 242, and 244 (of a plurality of electrodes), and a second coil 262 includes electrodes 250, 252, and 254 (of the plurality of electrodes), although each of the coils may include more or fewer electrodes. For example, each coil may include between one and six electrodes, preferably at least two electrodes, and more preferably, three electrodes. As shown in FIG. 5, the electrodes are arranged only in the distal portion 230 of the lead body 210, with no electrodes being arranged in the proximal portion 220 or a transition portion 225. However, in other embodiments, an electrode may optionally be disposed on the transition portion 225. In the example of FIG. 5, the first electrode 240 preferably is fixed to the first coil 260 at about 270 to 315 degrees of the first coil 260. The electrodes on each coil (e.g., the electrodes 240, 242, and 244 on the first coil 260) preferably are equally spaced apart from each other. For example, the electrodes 240, 242, and 244 may be spaced apart by about 2 mm to 10 mm. The example of FIG. 5 may differ from the example of FIG. 1, in part, based on the approximate location of the first electrode 240 on the first coil 260, the spacing between the electrodes 240, 242, and 244 of the first coil 260 and/or the spacing between the electrodes 250, 252, and 254 of the second coil 262. As illustrated in FIG. 5, all of the plurality of electrodes in each coil may be disposed on only a portion of the coil, such as one half or one quarter of the circumference of the coil to aid in positioning electrodes more proximate an adjacent phrenic nerve.

FIGS. 6-10 depict examples of leads in which a helix includes three coils. As described herein, when the helix includes a third coil in addition to first and second coils as described above, the third coil may provide a stabilization function. The third coil may be provided without electrodes and may serve, in part, a stabilization function that is especially useful during implantation of the lead. This stabilization coil may be located anywhere in the helix provided in the distal portion of the lead body, for example, as a first or most proximal coil of the helix, as a last or most distal coil of the helix, or as a middle coil of the helix.

As described herein, when the helix in the distal portion of the lead body includes at least three coils, it is preferable to provide at least two of the coils with electrodes, and an additional coil without electrodes, where the additional coil provides a stabilization function, and is referred to herein as a “stabilization coil.” In particular, use of the stabilization coil can improve implantation of the lead, whereby the lead can be placed at a desired location in the vein, thus increasing stability as compared to a helix having fewer than three coils.

Referring to FIG. 6, a lead 300 has a lead body 310 including a proximal portion 320 that is generally straight, and a distal portion 330 formed with a helix, where the distal portion 330 (i.e., the helix) may be formed with a predetermined bias. As with the leads 100, 200 illustrated in FIGS. 1 and 5, a central axis 332 of the helix may be approximately coaxial with a longitudinal axis 322 of the lead body 310. As similarly discussed above, the longitudinal axis 322 of the lead body 310 may deviate from the central axis 332 of the helix by an angle α such that the two axes are approximately coaxial. As also similarly discussed above, the longitudinal axis 322 and the central axis 332 of the helix may be parallel or substantially parallel with one another, but slightly offset from one another, such that the longitudinal axis 322 lies within a cylinder 75 centered about the central axis 332 of the helix, and such that the two axes are approximately coaxial. In contrast to the leads shown in FIGS. 1 and 5, the distal portion 330 of the lead 300 illustrated in FIG. 6 is formed with a converging diameter helix in which more distal portions of the helix have a smaller diameter than the more proximal portions. Such a lead including a converging diameter helix may be preferred where the lead 300 is to be implanted into a lumen having a decreasing diameter. For example, the use of the converging diameter helical lead may be preferred where the distal portion of the lead is disposed in the SVC or in the left pericardiophrenic vein.

As shown in FIG. 6, a first coil 360 includes electrodes 340, 342, and 344 (of a plurality of electrodes), and a second coil 362 includes electrodes 350, 352, and 354 (of the plurality of electrodes), although each of the coils may include more or fewer electrodes. For example, each coil may include between one and six electrodes, preferably at least two electrodes, more preferably, three electrodes. As provided in FIG. 6, the electrodes are arranged only in the distal portion 330 of the lead body, with no electrodes being arranged in the proximal portion 320 or a transition portion 325. It should be appreciated that some embodiments may optionally include an electrode disposed on the transition portion 325. The electrodes on each coil (e.g., the electrodes 340, 342, and 344 on the first coil 360) preferably are equally spaced apart from each other. To aid in positioning electrodes more proximate a phrenic nerve, all of the plurality of electrodes may be disposed on only a portion of the coil, such as one half or one quarter of the circumference of the coil as shown in FIG. 6, although the present invention is not so limited.

In FIG. 6, the helix in the distal portion 330 includes a third coil 364 that provides a stabilization function, and thus may be referred to as a stabilization coil. This third coil 364 is the most distal coil of the helix and preferably does not include any electrodes. This stabilization coil may help impart a shape to the lead that assists an implanter with orienting the distal portion 330 of the lead body during implantation. The stabilization coil may also act as a secondary fixation element. It should be appreciated that the inclusion of a third coil 364 as the most distal coil of the helix may be used with leads having a substantially constant diameter, or a diverging diameter, as well as well as those with a converging diameter as depicted in FIG. 6.

Another example of a lead body 410 with a distal portion having a helix that includes a stabilization coil is shown in FIG. 7. The example of FIG. 7 is similar to FIG. 6, except for the position of the stabilization coil. In FIG. 6, the third coil 364 being a stabilization coil is located at a distal end of the helix in the distal portion 330 of the lead body 310, whereas in FIG. 7, a first coil 460 located at a proximal end of the helix is provided without electrodes and performs a stabilization function. As shown in FIG. 7, a second coil 462 includes electrodes 440, 442, and 444 (of a plurality of electrodes), and a third coil 464 includes electrodes 450, 452, and 454 (of the plurality of electrodes), although each of the coils may include more or fewer electrodes. In FIG. 7, similar to FIG. 6, the electrodes are arranged only in a distal portion 430 of the lead body, preferably with no electrodes being arranged in a proximal portion 420 or a transition portion 425. It should be appreciated that some embodiments may optionally include an electrode disposed on the transition portion 425. The electrodes on each coil (e.g., the electrodes 440, 442, and 444 on the first coil 460) preferably are equally spaced apart from each other. As with the leads 100, 200, and 300 shown in FIGS. 1, 5, and 6, a central axis 432 of the helix may be approximately coaxial with a longitudinal axis 422 of the lead 400. As similarly discussed above, the longitudinal axis 422 of the lead body 410 may deviate from the central axis 432 of the helix by an angle α such that the two axes are approximately coaxial. As also similarly discussed above, the longitudinal axis 422 and the central axis 432 of the helix may be parallel or substantially parallel with one another, but slightly offset from one another, such that the longitudinal axis 422 lies within a cylinder 75 centered about the central axis 432 of the helix, and such that the two axes are approximately coaxial. All of the plurality of electrodes may be disposed on only a portion of the circumference of the coil. It should be appreciated that leads featuring a stabilization coil as the first or most proximal coil of the helix may include a diverging helix, a substantially constant diameter helix, or a converging helix as shown in FIG. 7. Further, the leads as depicted in FIGS. 1, 5, 6, and 7 may have different sizes to accommodate implantation in different sized lumens in the same manner as the leads depicted in FIGS. 2F-1 to 2F-3.

FIGS. 8 and 9 are each examples of a lead 500, 600 in which a lead body 510, 610 has a distal portion 530, 630 with a diverging helix, i.e., a bias of the helix of the distal portion 530, 630 diverges or expands distally along the helix. In other words, in the diverging helices depicted in FIGS. 8 and 9, successive coils of the helix in a direction of the distal end 530, 630 of the lead body 510, 610 have an increasing diameter. In the leads illustrated in FIGS. 8 and 9, a central axis of the helix may again be approximately coaxial with a longitudinal axis of the lead body. As similarly discussed above, the longitudinal axis of the lead body 510 and/or 610 may deviate from the central axis of the helix by an angle α such that the two axes are approximately coaxial. As also similarly discussed above, the longitudinal axis and the central axis of the helix may be parallel or substantially parallel with one another, but slightly offset from one another, such that the longitudinal axis lies within a cylinder 75 centered about the central axis of the helix, and such that the two axes are approximately coaxial.

As shown in FIG. 8, electrodes 540, 542, and 544 (of a plurality of electrodes) are arranged on a first coil, and electrodes 550, 552, and 554 (of the plurality of electrodes) are arranged on a second coil, although the first and second coils may include more or fewer electrodes. For example, each coil may include between one and six electrodes, preferably at least two electrodes, more preferably, three electrodes. As shown in FIG. 8, the electrodes are arranged only in the distal portion 530 of the lead body 510, with no electrodes being arranged in a proximal portion or a transition portion, although the present invention is not so limited, and an electrode may optionally be disposed on the transition portion. The electrodes on each coil (e.g., the electrodes 540, 542, and 544 on the first coil) preferably are equally spaced apart from each other, and the spacing of the electrodes 540, 542, and 544 on the first coil may or may not be the same as the spacing of electrodes 550, 552, and 554 on the second coil. As shown, all of the plurality of electrodes on the distal portion 530 of the lead 500 may be disposed on only a portion of the circumference of the coil, such as a quarter or a half of the circumference of the coil. In FIG. 8, the helix in the distal portion 530 includes a third coil that may be referred to as a stabilization coil. In particular, the third coil preferably does not include any electrodes. Although the stabilization coil is depicted at a distal end of the distal portion 530, it is possible that the stabilization coil may be positioned at a proximal end of the distal portion 530, or as the middle of the three coils that are depicted in FIG. 8.

Referring to FIG. 9, the helix in the distal portion 630 includes a first coil (i.e., the most proximal coil of the distal portion 630) that is a stabilization coil. In particular, the first coil preferably does not include any electrodes. In the example shown in FIG. 9, the second and third coils of the helix are configured to provide stimulation. In particular, electrodes 640, 642, and 644 (of a plurality of electrodes) are arranged on a second coil, and electrodes 650, 652, and 654 (of the plurality of electrodes) are arranged on a third coil, although the second and third coils may include more or fewer electrodes. For example, the second and third coils each may include between one and six electrodes, preferably at least two electrodes, more preferably, three electrodes. As shown in FIG. 9, the electrodes are arranged only in the distal portion 630 of the lead body 610, with no electrodes being arranged in a proximal portion or a transition portion, although the present invention is not so limited. The electrodes on each of the second or third coils (e.g., the electrodes 640, 642, and 644 on the second coil) preferably are equally spaced apart from each other. The spacing between the electrodes arranged on the second coil need not be the same as the spacing between the electrode arranged on the third coil, and all of the plurality of electrodes on the distal portion 630 of the lead 600 may be distributed around a circumference of the coil, or disposed on only a portion of the circumference of the coil as shown in FIG. 9.

Embodiments in which the lead body has a diverging helix may be useful for implantation in the right brachiocephalic vein 80 and/or the SVC 90. Typically the SVC 90 has a larger diameter than the right brachiocephalic vein 80, so the diverging helix may be useful in implanting the lead based on the expected smaller diameter of the right brachiocephalic vein 80 and the relatively larger diameter of the SVC 90. The leads 500, 600 as depicted in FIGS. 8 and 9 may have different sizes in the same manner as the leads depicted in FIGS. 2F-1 to 2F-3.

FIG. 10 is an example of a lead 700 in which a lead body 710 has a converging helix, i.e., a bias of the helix of the distal portion 730 converges or contracts distally along the helix. In other words, in the converging helix depicted in FIG. 10, successive coils of the helix in a direction of a distal end of the lead body 710 have a decreasing diameter. As shown in FIG. 10, electrodes 740, 742, and 744 (of a plurality of electrodes) are arranged on a first coil. In the example depicted in FIG. 10, a second coil is a stabilization coil, and thus is provided without any electrodes. A third coil includes electrodes 750, 752, and 754 (of the plurality of electrodes). Of course, even though three electrodes are depicted on each of the first and third coils, the first and third coils may include more or fewer electrodes. The electrodes on each of the first and third coils (e.g., the electrodes 740, 742, and 744 on the first coil) preferably are equally spaced apart from each other. In addition, it is noted that FIG. 10 is an example of a lead in which the second or middle coil (among the first, second, and third coils) is the stabilization coil. Alternatively, instead of the second coil, either the first coil or the third coil could be provided as the stabilization coil. As a further alternative, the second coil could replace either the first coil or the third coil as the stabilization coil in other embodiments, e.g., in examples in which the bias of the distal portion is formed with a constant or diverging helix. Similar to the lead 100 of FIG. 1, a central axis of the helix may be approximately coaxial with a longitudinal axis of the lead body 710. As similarly discussed above, the longitudinal axis of the lead body 710 may deviate from the central axis of the helix by an angle α such that the two axes are approximately coaxial. As also similarly discussed above, the longitudinal axis and the central axis of the helix may be parallel or substantially parallel with one another, but slightly offset from one another, such that the longitudinal axis lies within a cylinder 75 centered about the central axis of the helix, and such that the two axes are approximately coaxial.

Embodiments in which the lead body has a converging helix may be useful for implantation in cases in which the right brachiocephalic vein 80 has a varying diameter. The leads as depicted in FIGS. 8-10 may have different sizes in the same manner as the leads depicted in FIGS. 2F-1 to 2F-3. In addition, it is possible to select from a lead in which a lead body has a helix with coils of a constant diameter, a diverging helix, or a converging helix depending on the corresponding size of a lumen in which the lead is configured to be implanted.

As noted previously, anatomy varies from patient to patient, including the size and shape of a patient's vasculature. Variations in the diameter and shape of a patient's vascular lumen or lumens can make lead implantation and fixation challenging. The leads of the present disclosure provide advancements in implantation and fixation by customizing lead size and shape to provide a plurality of leads of different shapes and sizes corresponding to a variety of patient anatomies.

To illustrate the advantages of the presently described leads, a method of selecting an appropriate patient-specific lead for implanting into a specific patient is now described. The method described with respect to FIG. 11 may be performed by a physician, a surgeon, or other trained medical professional. In step S10, venous imaging may be conducted to assess the patient's vasculature (e.g., the shape and size of the patient's veins). The venous imaging may utilize x-ray technology, for example fluoroscopy, although other imaging techniques may alternatively be used, such as ultrasound, computerized tomography (CT), or magnetic resonance imaging (MRI). In one example, a discrete measurement may be made of a patient's right brachiocephalic vein and/or superior vena cava using a venogram and scaling capabilities of the fluoroscopic equipment. Alternatively, a calibrated comparison can be made by using a known feature in the fluoroscopic field of a particular size (e.g., a catheter or introducer sheath) to compare a vein with the known feature of the particular size so as to scale the vessel diameter. In other examples, it is possible to use intravascular ultrasound (IVUS) or other types of ultrasound instead of a venogram.

After performing the measurement of a patient's veins using either imaging technique, in step S20, a physician and/or other trained medical professional may evaluate or assess the venous and/or patient characteristics (e.g., patient height, weight, age, etc.). For example, the evaluation or assessment may reveal that the diameter of the right brachiocephalic vein is smaller than that of the superior vena cava. The assessment may also reveal that a confluence of the superior vena cava and the right brachiocephalic veins may have a large diameter as compared to the diameter of the right brachiocephalic vein alone.

After ascertaining the shape and size of a patient's vasculature in combination with the appropriate patient characteristics, a physician or other trained medical professional may determine, in step S30, a plan for implantation of the lead. The plan for implantation generally includes selecting the implantation location and/or vein(s) and determining the shape and size of a patient-specific lead based on the shape and size of the patient's vasculature at the implantation location. In some examples, the plan for implantation may include the lumen (or lumens) in which the lead is to be implanted, the location in the lumen(s) at which the distal portion of the lead is to be positioned, the size of the lead (e.g., small helical diameter, medium helical diameter, or large helical diameter) and the length of the lead to be implanted, whether the distal portion of the lead has substantially constant or even diameter coils, diverging coils, or converging coils, etc. For example, the implantation plan may determine that the appropriate patient-specific lead is to be implanted within the confluence of the superior vena cava (SVC 90 in FIGS. 2E and 4) and the right brachiocephalic vein 80. Positioning in the confluence of the SVC 90 and the right brachiocephalic vein 80 allows positioning a portion of the helices in the right brachiocephalic vein 80 for improved stability or fixation.

Where the lead is to be positioned at the confluence, the physician or other trained medical professional may select, at step S40, an appropriate sized lead, for example a medium or large diameter lead, with evenly sized coils, diverging coils, or converging coils, wherein the smallest coil(s) are placed in the right brachiocephalic vein 80. Any such implantation may also position the electrodes of the lead adjacent the phrenic nerve. For example, where it is desired to place the lead at a confluence of the of the SVC 90 and the right brachiocephalic vein 80, a lead with diverging coils may be selected at step S40 so that the larger diameter helices are disposed in the larger diameter SVC 90, and the smaller diameter helices are disposed in the right brachiocephalic vein 80. Such a lead may be similar to that illustrated in FIG. 8 or 9. In another alternative implantation, a physician or other trained medical professional may use a diverging lead to position the coils of the helix in the superior vena cava, for example, in cases where the SVC/brachiocephalic junction is wide.

Alternatively, if placement at the confluence of the SVC 90 and the right brachiocephalic vein 80 is not optimal, and the implantation plan determines that placement in the right brachiocephalic vein 80 is desired, the physician or other trained medical professional may select, at step S40, a small or medium diameter lead. The diameter may be selected to correspond to the size, particularly the diameter, of the patient's vasculature obtained in step S20. In certain patients, the right brachiocephalic vein 80 is larger, and may have a diameter similar in dimension to that of the SVC 90, in which case a large diameter lead may be appropriate. By implanting a lead of an appropriate size in the SVC 90 and/or the right brachiocephalic vein 80, it is possible to achieve more stable implantation and position electrodes of the lead to stimulate the right phrenic nerve from locations within these lumens that are preferably adjacent to the phrenic nerve. In certain cases a physician or trained medical professional may use a converging or diverging lead for implantation in the right brachiocephalic vein to obtain improved positioning and fixation, for example, in cases where the patient's right brachiocephalic vein is longer than average.

As discussed in more detail below, to aid in the selection of an appropriate patient-specific lead at step S40, a lead kit may be provided that includes a plurality of differently sized and shaped leads from which the physician or other trained medical professional may select the appropriate patient-specific lead. The lead kit may be provided such that each lead in the kit is individually packaged within a packaged lead kit, such that if not selected, may be used with a different patient thereafter. After selecting an appropriate patient-specific lead at step S40, the physician or other trained medical professional proceeds to step S50, wherein the physician or other medical trained professional proceeds with implantation.

Having selected an appropriate patient-specific lead, the sleep apnea system may then be implanted in the patient. The method of implanting the sleep apnea system may include implanting a medical electrical lead in the patient, and implanting an implantable pulse generator in the patient. Implanting the medical electrical lead may generally include steps of inserting the lead in a lumen (e.g., the right brachiocephalic vein), and positioning the lead within the lumen such that a plurality of electrodes (i.e., electrodes arranged along a helix of a distal portion of a lead body) are disposed against an internal wall of the lumen, and thus are configured to stimulate an adjacent nerve (e.g., the right phrenic nerve).

Referring to FIG. 12, a method for implanting a lead may be carried out using the lead of any one of FIGS. 1 and 2A-2D, or any one of the leads depicted in FIGS. 5-10, in order to enable stimulation of the right phrenic nerve. In step S110, a target lumen (e.g., the right brachiocephalic vein) is located. This step may be carried out by gaining venous access using the right axillary, cephalic or subclavian vein, and selecting a puncture site near the lateral border of the first rib when utilizing a subclavian approach while avoiding penetrating the subclavius muscle. Subsequently, in step S120, the lead is inserted in the right brachiocephalic vein, and in step S130, the lead is positioned in the vein with electrodes against an internal wall of the vein. The above steps S120 and S130 may include selecting the appropriate patient-specific stimulation lead model for the diameter of the brachiocephalic vein (e.g., step S40 of FIG. 11), ensuring a stylet is fully advanced within the lead to the distal end of the vein before inserting the lead, placing the lead through an introducer sheath and advancing the lead until the distal end of the lead is at the level of the superior vena cava or right atrium, retracting the stylet gradually and applying counterclockwise rotation to the lead body to allow the helical shape to form, applying gentle traction and counterclockwise rotation as needed to position the electrodes along the lateral wall of the vein, and assessing the stability of the lead position by requesting that the patient breathe deeply or cough during fluoroscopic observation.

After the lead is inserted in the lumen, positioned with electrodes against the lumen wall, and secured, the lead is tested for phrenic nerve capture (step S140). To test for capture, once the stimulation lead has been placed in the desired location, the stylet, or alternatively a guide wire, is retracted sufficiently to expose lead bias, if applicable, allowing the lead to engage the vein in a natural way. Then, an electrode testing configuration (cathode-anode electrode pair) is selected, and the stimulation is delivered. The testing may be provided using an external stimulation source. A single test pulse or multiple test pulse(s) may be delivered, and subsequently, the stimulation current may be increased or decreased incrementally as needed until a moderately strong diaphragmatic contraction is observed by means of abdominal palpation or fluoroscopic visualization. Each electrode of the plurality of electrodes disposed on the lead may be tested to determine that all are functionally operable, and which of the plurality of electrodes may be preferred for use over others. During this testing, each electrode of the plurality of electrodes, for example acting as a cathode, may be stimulated by the external stimulation source, with a surgical clip attached to the patient's body or other connection acting as the anode Each electrode may be ranked according to which provides the best capture of the phrenic nerve. Thereafter, the introducer sheath is removed prior to securing the lead. To secure the lead, the first ligature sleeve is positioned immediately proximal to the point of venous access and secured to the lead using permanent, non-absorbable sutures, where the ligature sleeve is anchored to the fascia or other suitable subcutaneous tissue using permanent, non-absorbable sutures. A second suture sleeve should be secured a minimum of 10 cm proximal to the first suture sleeve with a strain relief loop between the first and second sleeve for stability. Finally, the guide wire or stylet should be maintained within the lumen of the stimulation lead while securing the ligature sleeve to the lead body and anchoring to tissue in order to prevent damage to the stimulation lead insulation and conductor coil.

In step S150, a pocket is created for receiving the IPG, and then the IS-1 terminal pins of the lead are inserted in respective connector ports of the IPG, and set screws are tightened. Subsequently, the IPG is secured in the pocket while being aligned vertically with the pocket, preferably in a tight-forming manner.

As discussed above, to aid in the selection of an appropriate patient-specific lead at step S40 in FIG. 11, a lead kit may be provided that includes a plurality of differently sized and shaped leads from which the physician or other trained medical professional may select the appropriate patient-specific lead. Such a lead kit 1000 is illustrated in FIG. 13. As shown in FIG. 13, the lead kit 1000 may include a plurality of leads, The plurality of leads may include leads similar in structure and design to the substantially constant helical diameter two coil lead 100 of FIG. 1, the converging helical diameter three coil lead 300 of FIG. 6, and the diverging helical diameter three coil lead 500 of FIG. 8. The plurality of leads in the lead kit 1000 may all be of the same diameter d1, d2, d3, (see FIG. 2F-1 through 2F-3) and several kits of different diameters (e.g., a kit with different types of leads of diameter d1, another kit with different types of leads of diameter d2, etc.) may be provided, such the physician or other trained medical professional simply selects the kit with the appropriate size, and then selects from that kit the most appropriate lead for the patient, given the patient's characteristics and the planned implantation site. It should be appreciated that lead kits in accordance with aspects of the present disclosure are not limited to the leads illustrated in FIGS. 1, 6, and 8, as other kits may include different types of leads. For example, kits may be provided with a plurality of leads similar to the lead 100 of FIG. 1, or the lead 300 of FIG. 6, but in different diameters such as illustrated in FIG. 2F-1 through 2F-3.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the terms “right”, “left”, “top”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

The methods, systems, and devices discussed above are examples. Various alternative configurations may omit, substitute, or add various procedures or components as appropriate. Configurations may be described as a process which is depicted as a flow diagram or block diagram. Although each may describe the operations as a sequential process, many of the operations can be performed in parallel or concurrently. In addition, the order of the operations may be rearranged. A process may have additional stages not included in the figure. Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional stages or functions not included in the figure. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory processor-readable medium such as a storage medium. Processors may perform the described tasks.

Having described several example configurations, various modifications, alternative constructions, and equivalents may be used without departing from the disclosure. For example, the above elements may be components of a larger system, where other rules may take precedence over or otherwise modify the application of the invention. Also, a number of operations may be undertaken before, during, or after the above elements are considered. Also, technology evolves and, thus, many of the elements are examples and do not bound the scope of the disclosure or claims. Accordingly, the above description does not bound the scope of the claims. Further, more than one invention may be disclosed.

1-119. (canceled)

120. An implantable medical electrical lead comprising:
a lead body defining a longitudinal axis, the lead body having a proximal portion and a distal portion, the proximal portion being generally straight, and the distal portion being formed with a helix, the helix having a central axis being defined through the helix; and
a plurality of electrodes arranged along the helix, each of the electrodes having a length parallel to a lead body portion on which each of the electrodes is disposed,
wherein the longitudinal axis and the central axis are approximately coaxial.

121. The implantable medical electrical lead of claim 120, wherein the longitudinal axis diverges from the central axis by an angle α such that the longitudinal axis and the central axis are approximately coaxial.

122. The implantable medical electrical lead of claim 121, wherein a is less than 25 degrees.

123. The implantable medical electrical lead of claim 120, wherein the longitudinal axis and the central axis are substantially parallel to one another and displaced from one another by less than a radius of a coil of the helix such that the longitudinal axis and the central axis are approximately coaxial.

124. The implantable medical electrical lead of claim 120, wherein the lead body further comprises a transition portion arranged between the proximal portion and the distal portion of the lead body, the transition portion having a radius of curvature of about 9 mm to 11 mm.

125. The implantable medical electrical lead of claim 124, wherein the transition portion has a length along the central axis of about 20 mm.

126. The implantable medical electrical lead of claim 125, wherein the transition portion comprises a proximal segment comprising a first curve in a first plane, a distal segment comprising a second curve in a second plane, and an intermediate segment disposed between the proximal segment and the distal segment and comprising a complex curve in multiple planes.

127. The implantable medical electrical lead of claim 126, wherein the helix comprises a plurality of coils and wherein the radius of curvature of the transition portion is not less than a radius of curvature of a smallest coil of the helix.

128. The implantable medical electrical lead of claim 121, wherein the at least two coils of the helix are preformed to have one of a substantially constant diameter, an increasing diameter in a direction of a distal end of the lead body, or a decreasing diameter in the direction of the distal end of the lead body.

129. The implantable medical electrical lead of claim 120, wherein the helix comprises at least two coils.

130. The implantable medical electrical lead of claim 129, wherein the lead is one of a small lead having a helical diameter of about 14 to 18 mm, a medium lead having a helical diameter of about 19 to 23 mm, or a large lead having a helical diameter of about 24 to 28 mm.

131. The implantable medical electrical lead of claim 120, wherein the lead body further comprises a transition portion arranged between the proximal portion and the distal portion of the lead body.

132. The implantable medical electrical lead of claim 131, wherein the transition portion is connected to a first coil of a plurality of coils of the helix of the distal portion.

133. The implantable medical electrical lead of claim 131, wherein the transition portion has a radius of curvature of about 9 mm to 11 mm.

134. The implantable medical electrical lead of claim 133, wherein the transition portion has a length along the central axis of about 20 mm.

135. The implantable medical electrical lead of claim 134, wherein the transition portion comprises a proximal segment comprising a first curve in a first plane, a distal segment comprising a second curve in a second plane, and an intermediate segment disposed between the proximal segment and the distal segment and comprising a complex curve in multiple planes.

136. The implantable medical electrical lead of claim 135, wherein the helix comprises a plurality of coils and wherein the radius of curvature of the transition portion is not less than a radius of curvature of a smallest coil of the helix.

137. The implantable medical electrical lead of claim 136, wherein at least one of the electrodes is arranged in the transition portion.

138. The implantable medical electrical lead of claim 120, wherein the helix is preformed with at least a first coil and a second coil.

139. The implantable medical electrical lead of claim 138, wherein the first coil includes at least two electrodes of the plurality of electrodes.

140. The implantable medical electrical lead of claim 139, wherein the second coil includes at least two additional electrodes of the plurality of electrodes.

141. The implantable medical electrical lead of claim 120, wherein the helix is preformed with at least a first coil, a second coil, and a third coil.

142. The implantable medical electrical lead of claim 141, wherein the first coil includes at least two electrodes of the plurality of electrodes.

143. The implantable medical electrical lead of claim 142, wherein the second coil includes at least two additional electrodes of the plurality of electrodes.

144. The implantable medical electrical lead of claim 143, wherein the third coil is configured as a stabilization coil without including any electrodes.

145. The implantable medical electrical lead of claim 141, wherein the first coil is configured as a stabilization coil without including any electrodes.

146. The implantable medical electrical lead of claim 145, wherein the second coil includes at least two electrodes of the plurality of electrodes.

147. The implantable medical electrical lead of claim 146, wherein the third coil includes at least two additional electrodes of the plurality of electrodes.

148. The implantable medical electrical lead of claim 141, wherein the first coil includes at least two electrodes of the plurality of electrodes, wherein the third coil includes at least two additional electrodes of the plurality of electrodes, and wherein the second coil is configured as a stabilization coil without including any electrodes.
